(19) United States
(12) Patent Application Publication
(10) Pub. No.: US 2004/0161745 A1
(43) Pub. Date: Aug. 19, 2004

(54) Human heparanase-related polypeptide and nucleic acid

(76) Inventors: Gerhard Siemeister, Berlin (DE); Bertram Weiss, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/168,795

(22) PCT Filed: Dec. 18, 2000
(86) PCT No.: PCT/EP00/12909

(30) Foreign Application Priority Data
Dec. 23, 1999 (EP) 99125831.0

(51) Int. Cl. C12Q 1/68; C08B 37/10; C07H 21/04; C12N 9/24

(52) U.S. Cl. 435/6; 435/69.1; 435/200; 435/320.1; 435/325; 530/388.26; 536/23.2; 536/21

(57) ABSTRACT

The present invention relates to newly identified polynucleotides, and polypeptides encoded by such polynucleotides, the use of such polypeptides, as well as the production of such polynucleotides and polypeptides. More particularly, a polypeptide of the present invention is a heparanase-related endoglucuronidase. The invention also relates to vectors and host cells comprising a polynucleotide of the invention. Furthermore, the invention relates to antibodies directed to polypeptides according to the present invention and to pharmaceutical compositions and diagnostic reagents comprising such antibodies, polypeptides or polynucleotides. The invention further relates to a method of altering, modifying or otherwise modulating the level of expression of the heparanase-related endoglucuronidase in a cell or in a organism. A further aspect of the invention are assay systems suitable for identifying modulators, e.g. agonists or antagonists of such polypeptides.

### FIELD OF THE INVENTION

The present invention relates to newly identified polynucleotides, and polypeptides encoded by such polynucleotides, the use of such polypeptides, as well as the production of such polynucleotides and polypeptides. More particularly, a polypeptide of the present invention is a heparanase-related endoglucuronidase. The invention also relates to vectors and host cells comprising a polynucleotide of the invention. Furthermore, the invention relates to antibodies directed to polypeptides according to the present invention and to pharmaceutical compositions and diagnostic reagents comprising such antibodies, polypeptides or polynucleotides. The invention further relates to a method of altering, modifying or otherwise modulating the level of expression of the heparanase-related endoglucuronidase in a cell or in a organism. A further aspect of the invention are assay systems suitable for identifiying modulators, e.g. agonists or antagonists of such polypeptides.

### BACKGROUND OF THE INVENTION

Extracellular matrix (ECM) and basement membrane (BM) proteins are embedded in a fibre meshwork consisting mainly of heparan sulfate proteoglycan (HSPG). HSPG 's are prominent compounds of blood vessels (subendothelial basement membrane) which support the endothelial cells and stabilize the structure of the capillary wall. Expression of heparanase, an endo-β-D-glucuronidase, in platelets, placental trophoblasts, and leucocytes demonstrates the normal function of heparanase in embryonic morphogenesis, wound healing, tissue repair, and inflammation. In concert with ECM-digesting proteases heparanase enables cells to traverse the basement membrane and releases heparin-binding growth factors (e.g. bFGF, VEGF) which are stored in the ECM (Finkel et al., Science 285 (1999), 33-34; Eccles, Nature Med. 5 (1999), 735-736).

Heparanase, which has recently been cloned by 4 independent groups (Vlodavsky et al., Nature Med. 5 (1999), 793-802; Hulett et al., Nature Med. 5 (1999), 803-809; Toyoshima and Nakajima, J. Biol. Chem. 274 (1999), 24153-24160; Kussie et al., Biochem. Biophys. Res. Comm. 261 (1999), 183-187), is expressed as a 65 kDa precursor protein which becomes N-terminally processed into the 50 kDa active enzyme. Recombinant expression of the active enzyme has been demonstrated in CHO, NIH 3T3 and in COS-7 cells. Although several apparently different heparanase activities have been described previously, the 4 groups which cloned the heparanase cDNA from different sources (normal and tumor cells) reported on identical cDNA sequences.

Several lines of evidence demonstrate an involvement of ECM degrading glucuronidases in tumor growth and metastasis formation: (1) Heparanase was shown to be preferentialy expressed on the mRNA and the protein level in human tumor tissues as compared to the corresponding normal tissue, e.g. invasive ductal carcinoma of the breast, hepatocellular carcinoma, ovary adenocarcinoma; squamous carcinoma of the cervix, colon adenocarcinoma (Vlodavsky et al., supra). (2) Increased levels of heparanase were shown in sera and urine of metastatic tumor-bearing animals and in cancer patients (Vlodavsky et al., supra). (3) Heparanase mRNA expression and enzyme acitivity correlates with metastatic potential of human and rat breast tumor cell lines (Vlodavsky et al., supra; Hulett et al., supra). (4) Low metastatic tumor cells aquire a highly metastatic phenotype upon transfection of heparanase cDNA, e.g. shown for murine T lymphoma L5178Y and mouse B16-F1 melanoma (Vlodavsky et al., supra). (5) The sulfated oligosaccharide PI-88 (phosphomannopentaose SO₄), which inhibits heparanase activity, inhibits primary tumor growth, metastasis formation, and tumor vascularization (Parish et al., Cancer Res. 59 (1999), 3433-3441).

### SUMMARY OF THE INVENTION

The present invention provides a new isolated nucleic acid molecule comprising a sequence of nucleotides encoding or complementary to a sequence encoding a polypeptide having endoglucuronidase enzymatic activity or a fragment thereof.

The present invention further relates to a polypeptide encoded by the polynucleotide, a functional fragment or a functional derivative or a functional analog thereof.

Another aspect of the invention relates to a process for preparing such a polypeptide or such a polynucleotide.

A further aspect of the invention relates to a recombinant vector comprising such a polynucleotide, preferably in operative linkage to an expression control sequence and a host cell transformed with such a recombinant vector.

Moreover, the present invention relates to a method of altering, modifying or otherwise modulating the level of expression of such a polypeptide or such a polynucleotide in a cell or in a organism.

Another aspect of the present invention relates to a method of diagnosis utilizing such a polynucleotide, or fragment or derivative thereof, or polypeptide, or fragment or derivative thereof.

Furthermore, the present invention relates to antibodies specifically recognizing and binding to such a polypeptide and to a method of diagnosis utilizing such an antibody.

Moreover, the present invention relates to pharmaceutical compositions comprising such a polynucleotide or such a polypeptide or such an antibody or a fragment thereof, and to a method of treatment comprising administration of such a polynucleotide or polypeptide or antibody or a fragment thereof.

A yet further aspect of the present invention relates to a method for identifying a substance capable of modulating the biological activity of such a polypeptide, and substances obtainable by such a method.

### DETAILED DESCRIPTION OF THE INVENTION

An isolated nucleic acid molecule comprising a nucleotide sequence encoding or complementary to a sequence encoding a polypeptide having the enzymatic activity of an endoglucuronidase is provided.

In a preferred embodiment thereof an isolated nucleic acid molecule according to the present invention is the nucleic acid molecule comprising (a) at least the protein coding portion of the nucleotide sequence set forth in SEQ ID NO 1, (b) a nucleotide sequence corresponding to the sequence 25 of (a) in the scope of the degeneracy of the genetic code or (c) a nucleotide sequence hybridizing under stringent conditions to the nucleotide sequence of (a) and/or (b).

The present invention further provides a polypeptide encoded by the nucleic acid molecule according to the present invention. Preferably, the polypeptide comprises (a) the amino acid sequence set forth in SEQ ID NO 2 or (b) an amino acid sequence having an identity of at least 70%, preferably at least 85% and more preferably at least 95% to the amino acid sequence of (a).

In addition to the nucleotide sequence as set forth in SEQ ID NO 1 and a nucleic acid sequence corresponding thereto in the scope of the degeneracy of the genetic code, the present invention encompasses also a nucleotide sequence which hybridizes under stringent conditions with one of the sequences as defined above. The term “hybridization under stringent conditions” according to the present invention is defined according to Sambrook et al., Molecular Cloning, A Laboratory Manual, Cold Spring Harbor Laboratory Press (1989), 1.1.01-1.104. Preferably, hybridization under stringent conditions means that after washing for 1 h with 1×SSC and 0.1% SDS at 50° C., preferably at 55°C., more preferably at 62° C. and most preferably at 68° C., particularly for 1 h in 0.2×SSC and 0.1% SDS at 50° C., preferably at 55° C., more preferably at 62° C. and most preferably at 68° C. a positive hybridization signal is observed. A nucleotide sequence which hybridizes under the above washing conditions with the nucleotide sequence as set forth in SEQ ID NO 1 or a nucleotide sequence corresponding thereto in the scope of the degeneracy of the genetic code is encompassed by the present invention.

Preferably, the nucleotide sequence according to the invention is a DNA, e.g. a cDNA, genomic DNA or synthetic DNA, which may be double-stranded or single-stranded, and if single-stranded may be the coding or non-coding (anti-sense) strand. It can, however, comprise an RNA, e.g. an mRNA, pre-mRNA and synthetic RNA either the coding or the non-coding (anti-sense) strand or a nucleic acid analog such as a peptidic nucleic acid. Particularly preferred, the nucleotide sequence according to the invention comprises a protein coding portion of the nucleotide sequence shown in SEQ ID NO 1 or a sequence, having an identity of more than 70%, preferably more than 85% and particularly preferred more than 95% of the nucleotide sequence shown SEQ ID NO 1 or a portion thereof having a length of preferably at least 20 nucleotides, particularly at least 30 nucleotides and most preferably at least 50.

The identity is determined on nucleotide or protein level as follows:

I=n:L,

wherein

I represents the identity in percent

n represents the number of different nucleotides or amino acids between a test sequence and a basic sequence selected from the nucleotide sequence of SEQ ID NO 1, the amino acid sequence SEQ ID NO 2 or a portion thereof, respectively and

L is the length of the basic sequence to be compared with a test sequence.

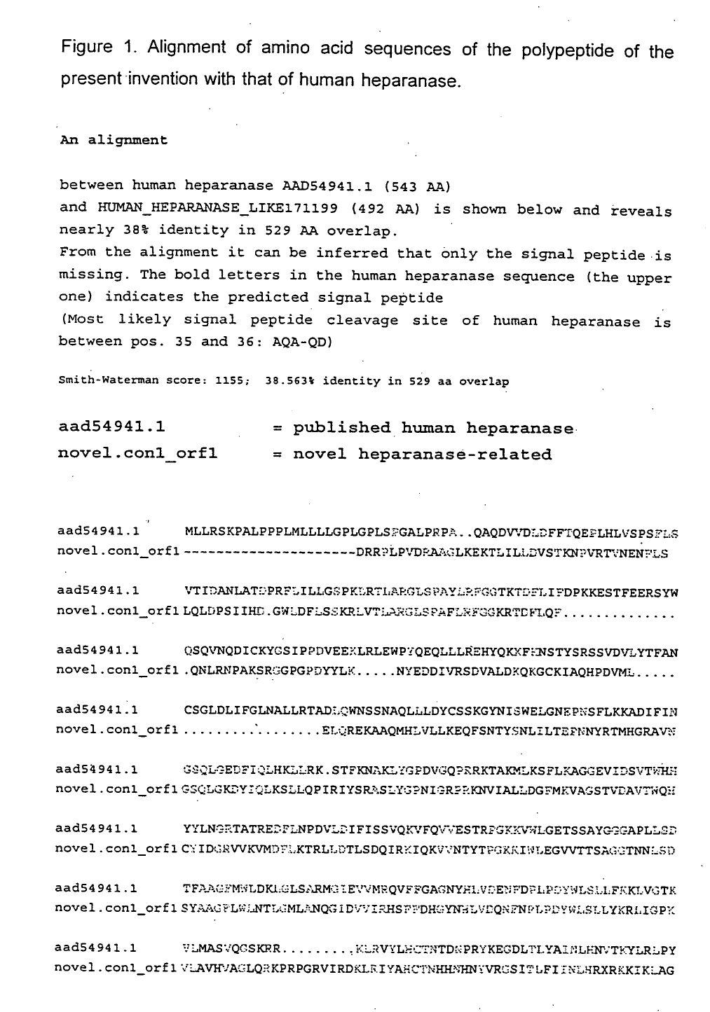

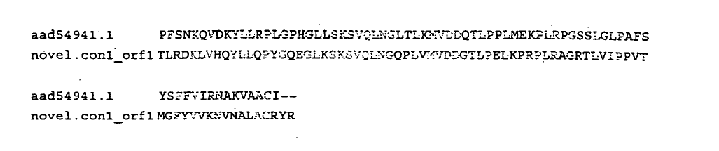

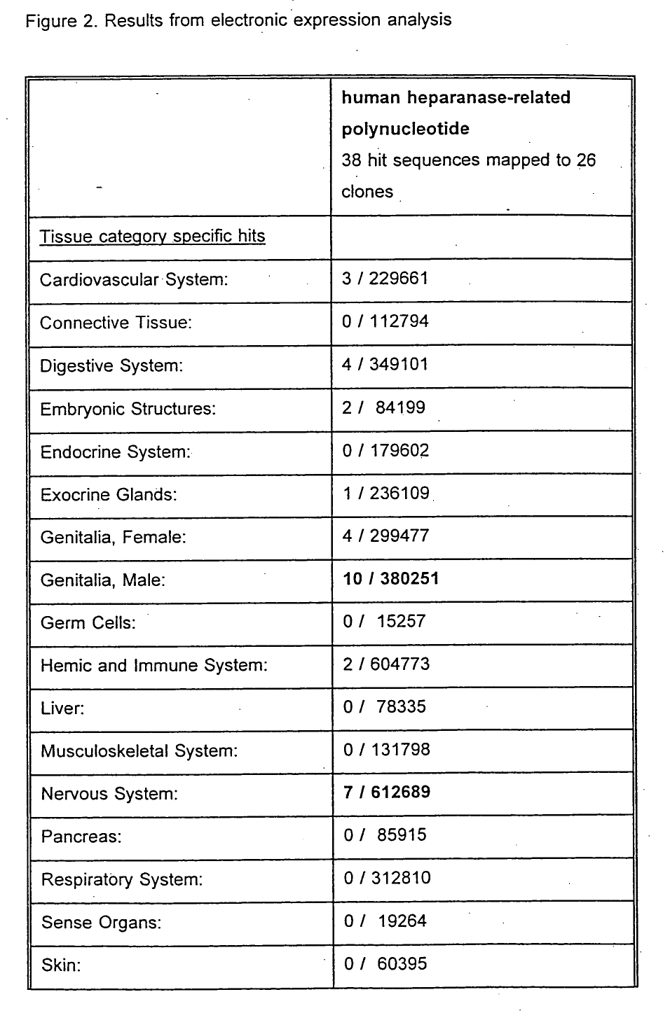

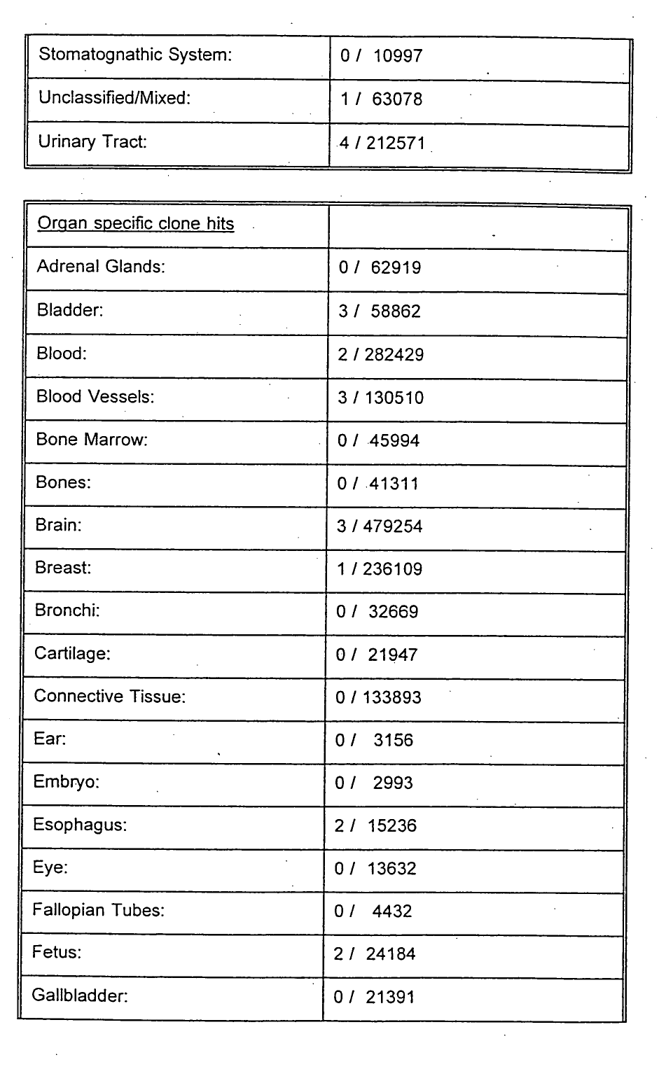

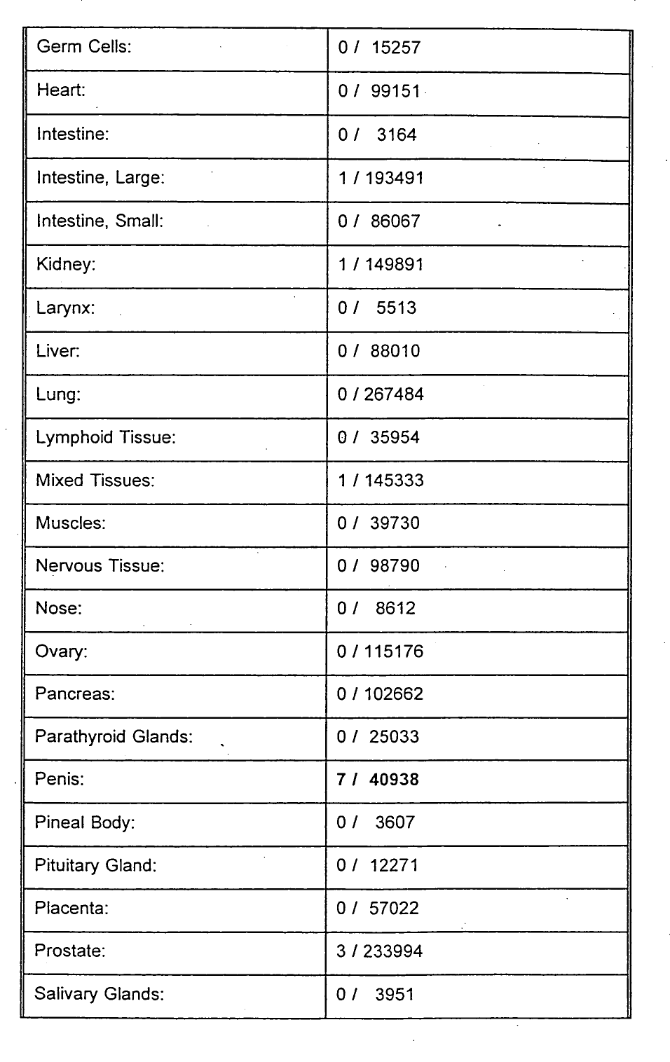

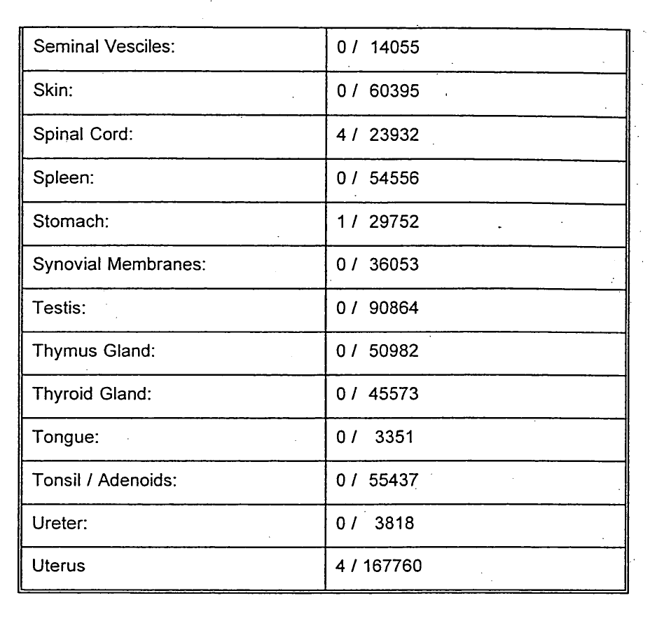

A polynucleotide of the present invention may be obtained from mammalian, e.g. human cells or from a cDNA library or a genomic library derived from mammalian, e.g. human cells. In particular, the polynucleotide described herein may be isolated from cDNA libraries (PENCNOTO7, BLADNOTO9, PROSTUTO8, BRSTNOT27, MIXDNOPO1, ESOGNOTO4, PENCNOTO3) available from Incyte Inc. The cDNA insert shown in SEQ ID NO 1 is 3943 base pairs (bp) in lenght and contains an open reading frame encoding a protein 492 amino acids in lenght. The predicted amino acid sequence of the polypeptide of the present invention shares 38% identical amino acids with human heparanase (FIG. 1). The 5′-end of the cDNA of the present invention is incomplete; the predicted mature protein is complete as inferred from homology to human heparanase. Electronic expression (Northern) analysis implicates preferential expression of the polynucleotide of the present invention in nervous system and male genitalia tissues (FIG. 2).

The present invention further relates to variants of the herein described 5 polynucleotide which code for fragments, analogs and derivatives of the polypeptide having the deduced amino acid sequence of SEQ ID NO 2. The present invention also relates to polynucleotide probes constructed from the polynucleotide sequence of SEQ ID NO 1 or a segment of SEQ ID NO 1. Variants of the herein described polynucleotide include deletion variants, substitution variants and addition or insertion variants.

The present invention also includes polynucleotides, wherein the coding sequence for the polypeptide, or a segment thereof, may be fused in the same reading frame to a polynucleotide sequence which aids the expression is or secretion of a polypeptide from a host cell, or which allows the purification of the polypeptide of the present invention (i.e. a poly-histidin-tag, a hemagglutinin tag, a GST-tag).

A process for the preparation of a polynucleotide according to the present invention represents an aspect of the present invention. Such a process may comprise chemical synthesis, recombinant DNA technology, polymerase chain reaction or a combination of these methods. Preferably the polynucleotide is obtained by means of an amplification reaction, e.g. a PCR using sequence-specific oligonucleotide primers, from a suitable source as described above.

The polypeptide of the present invention may be a recombinant polypeptide, a natural polypeptide or a synthetic polypeptide. The functional fragment, derivative or analog of the present invention may be one in which one or more amino acids are substituted with another amino acid, or one in which one or more of the amino acid residues includes a substituent group, or one in which the polypeptide is fused with another compound (i.e. polyethylene glycol), or one in which additional amino acids are fused to the polypeptide (i.e. a leader sequence, a secretory sequence, a purification tag).

The present invention also relates to a recombinant vector comprising a polynucleotide of the present invention. Preferably, such a vector is an expression vector, i.e. a vector comprising the polynucleotide of the present invention operatively linked to a suitable expression control sequence. The vector may be a prokaryotic or eukaryotic vector. Examples of prokaryotic vectors are chromosomal vectors such a bacteriophages and extrachromosomal vectors such as plasmids, wherein circular plasmid vectors are particulary preferred. Suitable prokaryotic vectors are disclosed, e.g. in Sambrook et al., supra, Chapters 1-4. On the other hand, the vector may be a eukaryotic vector, e.g. a yeast vector or a vector suitable for expression in higher cells, e.g. insect cells, plant cells or vertebrate cells, particularly mammalian cells. Preferred examples of eukaryotic vectors are plasmids or viral vectors. Suitable eukaryotic vectors are disclosed in Sambrook et al., supra, Chapter 16.

Furthermore, the present invention relates to a cell which contains at least one heterologous copy of a polynucleotide or a vector as defined above. The polynucleotide or the vector may be inserted into the cell by known means, e.g. by transformation (this term also including transfection, electroporation, lipofection, infection etc.). The cell may be a eukaryotic or a prokaryotic cell. Methods for transforming cells with nucleic acids are generally known and need not be explained in detail. Examples for preferred cells are eukaryotic cells, particulary vertebrate and more particulary mamalian cells.

Another aspect of the present invention relates to a recombinant process for the preparation of a polypeptide of the present invention, said process comprising cultivation of a host cell transformed with a polynucleotide or a vector as described above under conditions suitable for performing expression of the polypeptide, and isolation of the thus-expressed polypeptide from the cell or from the culture supernatant. The host cells can be cultured in conventional nutrient media modified as appropriate for selecting transformants, amplifying the polynucleotide or the vector or purification of the polypeptide.

The thus-expressed polypeptide of the present invention may be recovered and purified from recombinant cell cultures by methods used heretofore, including detergent homogenates, Heparin-Sepharose chromatography, cation exchange chromatography, Con A-Sepharose chromatography, gel-filtration chromatography, Ni-chelating chromatography, glutathion-sepharose (agarose) chromatography, hydrophobic interaction chromatography, and antibody affinity chromatography.

A polypeptide of the present invention may be a purified product naturally expressed from a high expressing cell line, or a product of chemical synthesis, or produced by recombinant techniques from a prokaryotic or eukaryotic host. Depending on the host employed in a recombinant production procedure, a polypeptide of the present invention may be glycosylated or non-glycosylated.

Another aspect of the present invention relates to an oligonucleotide or a derivative thereof, which hybridizes under stringent conditions with the nucleotide sequence set forth in SEQ ID NO 1. Such an oligonucleotide may have a length of, e.g., from about 5, preferably from about 15 to about 100 or even several hundred nucleoside units or analogs thereof, depending on the intended use.

An oligonucleotide of the invention may be used as a cloning primer, or as a PCR primer, or as a sequencing primer, or as a hybridization probe. Another use relates to stimulating or inhibiting expression of a polypeptide of the present invention in vivo by the use of sense or anti-sense technology. These technology can be used to control gene expression through triple-helix formation on double-stranded DNA or anti-sense mechanisms on RNA, both of which methods are based on binding of such an oligonucleotide to DNA or RNA. Still another use of oligonucleotides, particularly RNA oligonucleotides relates to an expression control by using ribozyme technology. The oligonucleotides can be delivered to cells by procedures in the art either directly or such that the anti-sense or ribozyme RNA or DNA may be expressed in vivo to inhibit production of a polypeptide of the present invention. Anti-sense constructs or ribozymes to a polynucleotide of the present invention inhibit the action of a polypeptide of the present invention and may be used for treating certain disorders, for example, cancer and cancer metastasis.

Further, such oligonucleotides can be used to detect the presence or absence of a polynucleotide of the present invention and the level of expression of such a polynucleotide. Furthermore, such oligonucleotide can be used for the detection of mutations within the gene encoding the polypeptide of the present invention. Mutations within the gene may be correlated with disease or prognosis of disease. Therefore, such oligonucleotides are useful as diagnostic markers for the diagnosis of disorders such as cancer, cancer metastasis, and aberrant angiogenesis.

The polypeptides, their functional fragments, derivatives or analogs thereof, or a cell expressing them, or the polynucleotide or fragments thereof, can be used as an immunogen to produce antibodies thereto. Therefore, the present invention relates to an antibody which specifically recognizes and binds to a polypeptide of the invention.

Such an antibody can be, for example, a polyclonal or a monoclonal antibody. The present invention also includes chimeric, single chain and humanized antibodies, as well as Fab fragments. Various procedures known in the art may be used for the production of such antibodies and fragments.

Polyclonal antibodies may be obtained by immunizing experimental animals with suitable polypeptide or peptide antigens optionally coupled to a carrier and isolating the antibodies from the immunized animals. Monoclonal antibodies may be obtained by the hybridoma technique developed by Köhler and Milstein. Methods for generating polyclbnal and monoclonal antibodies, respectively, are generally known and need not be explained in detail (Harlow and Lane, Antibodies: A Laboratory Manual, Cold Spring Harbor Laboratory, 1988).

Such an antibody can be used for isolating the polypeptide from a tissue expressing that polypeptide. An antibody specific to a polypeptide of the present invention may further be used to inhibit the biological action of the polypeptide by binding to the polypeptide. In this manner, the antibodies may be used in therapy, for example to treat cancer. The cancer therapy may be carried out according to the protocols described by Weiner (Semin. Oncol. 26 (1999), 41-50) or references cited therein.

Further, such antibodies can detect the presence or absence of a polypeptide of the present invention and the level of concentration of such a polypeptide and, therefore, are useful as diagnostic markers for the diagnosis of disorders such as cancer, cancer metastasis, and aberrant angiogenesis.

In a further aspect, the present invention relates to a method for identifying a substance capable of modulating the biological activity or expression of a polypeptide of the present invention. Thus, the present invention is directed to a method for identifying antagonists and inhibitors, as well as agonists and stimulators of the function or activity or expression of a polypeptide of the present invention.

For example, an antagonist may bind to a polypeptide of the present invention and inhibit or eliminate its function. The antagonist, for example, could be an antibody or an high-affinity oligonucleotide or a peptide against the polypeptide which eliminated the glucuronidase activity of the polypeptide by binding to the polypeptide. An example of an inhibitor is a low molecular weight molecule which inactivates the polypeptide by binding to and occupying the catalytic site, thereby making the catalytic site inaccessible to a substrate, such that the biological activity of the polypeptide is prevented.

Antagonists and inhibitors may be used to treat cancer, cancer metastasis, and aberrant angiogenesis by preventing the polypeptide from functioning to break down heparan sulfate proteoglycan from extracellular matrix.

The antagonists and inhibitors identified by the method as described above or derivatives thereof may be employed in a composition with a pharmaceutical acceptable carrier.

In particular, the present invention relates to an assay for identifying the above-mentioned substances, e.g. low molecular weight inhibitors, which are specific to the polypeptides of the present invention and prevent them from functioning or prevent their expression. Either natural or synthetic carbohydrate substrates would be used to assess endo-glucuronidase activity of the polypeptide.

A further aspect relates to a polynucleotide or a polypeptide according to the present invention for use in medicine. In particular, the invention relates to the use of a polypeptide or a polynucleotide according to the present invention in the preparation of a pharmaceutical composition for the treatment of a disease resulting from shortage or lack of said polypeptide. Instead of or in addition to a polynucleotide or a polypeptide of the present invention, an agonist of the polypeptide or an expression inducer/enhancer of such a polypeptide may be used for the medicinal purposes. Such diseases are, for example, trauma, autoimmune diseases, skin diseases, cardiovascular diseases, and nervous system diseases. The polynucleotide of the present invention may be used in gene therapy. The gene therapy may be carried out according to protocols described by Beutler (Biol. Blood Marrow Transplant 5 (1999), 273-276) or Gomez-Navarro et al., (Eur. J. Cancer 35 (1999), 867-885) or references cited therein.

Another aspect relates to an antibody according to the present invention or a fragment thereof for use in medicine. In particular, the invention relates to the use of an antibody according to the present invention in the preparation of a pharmaceutical composition for the treatment of a disease resulting from excessive activity or overexpression of a polypeptide of the present invention. Instead of an antibody of the present invention, an antagonist or an inhibitor or an expression inhibitor of such a polypeptide may be used for the medicinal purposes. Such diseases are, for example, cancer, cancer metastasis, angiogenesis and inflammation including arthritis.

Furthermore, the invention is directed to a pharmaceutical composition suitable for administration to a warm-blooded animal inclusive man suffering from a disease resulting from shortage or lack or inactivity of a polypeptide of the present, invention, or suffering from a disease resulting from excessive activity or overexpression of a polypeptide of the present invention.

Since the polynucleotide of the present invention is preverentially expressed in male genitalia tissues modulation of expression and/or activity of the encoded polypeptide may be used for medicinal intervention in male genitalia function (i. e. male fertility control, erectile dysfunction).

### EXAMPLES

### Example 1

Identification of a Polynucleotide of the Present Invention

Using the published sequence of human heparanase (AAD 54941.1) three Incyte templates (i.e. assemblies of Incyte ESTs) could be identified to share significant homology to the human heparanase. Some of these ESTs of each template were ordered from Incyte. Determination of the nucleotide sequence of the 3′- and 5′-ends of each EST clone revealed more novel sequence information which lead to further two assemblies from Incyte clones. Combining this sequence information and sequence information from own sequencing efforts of these Incyte clones enabled us to assemble a novel paralogue, human heparanase-related polypeptide, of human heparanase. The novel sequence comprises 3943 bp and the identified coding sequence ranges from 1 bp - 1479 bp (including STOP codon). The 5′ end is still open as both coding region analysis (as determined by the program ESTSCAN) and homology to human heparanase suggest.

### Example 2

Electronic Expression Analysis

Based on the number of ESTs for a given tissue one can estimate or predict a measure for the in vivo expression level of the given gene in this given tissue.

“Electronic-northern” is a bioinformatic method that firstly identifies the overall number for all ESTs for a given tissue (so-called“pool-size”) that are in the database and secondly the number of ESTs from that tissue which correspond only to the query sequence.

This is done by a BLAST (NCBI BLAST v. 2.0.10; Altschul et al., Nucleic Acid Res. (1997) 25, 3389-3402) search using the cDNA of the gene of interest as query and the human EST database (LifeSeqGold from Incyte) as data source. The search parameters were E=1e-30. A SQL-query in the database retrieves then for each EST coming up from the search its tissue source and the pool-size for each tissue.

This data is believed to correlate with the expression level in vivo. Statistical analysis (normalisation on pool-size and confidence interval determination) helps here to estimate the reliability of the data and to compare the expression level between different tissues. The reliability of this prediction method increases usually with the number of hits/tissue and the pool-size of a tissue.

### Example 3

Expression of the Polynucleotide

The coding region of the polynucleotide given in SEQ ID NO 1 was amplified by PCR using 5′-primer HepR1 (5′-GAC AGG AGA CCC TTG CCT GTA GAC-3′) and 3′-primer HepR2 (5′-ATA GTC GAG TTA TCG GTA GCG GCA GGC CAA AGC-3′) and DNA isolated from clones #3207535H1 and #3385824H1 the database LifeSeqGold from Incyte Inc. issue of Oct/Nov 1999 as template DNA. The 1488 bp DNA was phosphorylated using T4 polynucleotide kinase followed by restriction digestion using Xhol. The fragment was ligated in frame into pISP-myc vector providing an N-terminal immune globuline signal sequence followed by an myc-tag epitope. Upon restriction digestion using HindII and Xhol the fragment was ligated into the appropriate sites of expression vector pCEP4 (Invitrogen) generating expression vector HepR-pCEP. HepR-pCEP was stably transfected into MCF7, MBA-231, and MBA-468 breast carcinoma cell lines, as well as in CHO cells. The recombinant protein was detected using an anti-myc-tag epitope antibody.

For expression in the insect cells, the PCR-fragment was released from pISP-myc vector using EcoRI and XbaI. The fragment was cloned into pVL1392 baculovirus transfer vector generating HepR-pVL vector and transfected into Sf9 insect cells.

### Example 4

Production of Antibodies

Polypeptide purified from infected Sf9 insect cells using expression vector HepR-pVL of example 3 was used for immunization of mice and rabbits, respectively, using standard procedures (Harlow and Lane, Antibodies: A Laboratory Manual, Cold Spring Harbor Laboratory, 1988).

1. A polynucleotide comprising
(a) the sequence as set forth in SEQ ID NO 1 or at least the protein coding portion thereof,
(b) a nucleotide sequence corresponding to the sequence of (a) in the scope of the degeneracy of the genetic code, or
(c) a nucleotide sequence hybridizing under stringent conditions with a sequence from (a) and/or (b).

2. The polynucleotide of claim 1 encoding a polypeptide having the biological activity of an endo-glucuronidase.

3. The polynucleotide of claim 1 or 2 having an identity of at least 70% to the nucleotide sequence as set forth in SEQ ID NO 1 or a fragment thereof.

4. The polynucleotide of any one of claims 1 to 3 which is a DNA, an RNA or a nucleic acid analog.

5. A recombinant vector comprising at least one copy of the polynucleotide of any one of claims 1-4.

6. The vector of claim 5 which is an expression vector.

7. A cell which is transformed with the polynucleotide of any one of claims 1-4 or with the vector of any one of claims 5-6.

8. A polypeptide which is encoded by the polynucleotide of any one of claims 1-4.

9. The polypeptide of claim 8 comprising
(a) the amino acid sequence as set forth in SEQ ID NO 2, or
(b) an amino acid sequence having a identity of at least 70% to the amino acid sequence of (a) or a fragment thereof.

10. The polypeptide of claim 8 or 9 having an endo-glucuronidase activity.

11. The polypeptide of any one of claims 8-10 or a fragment thereof being capable of eliciting specific antibodies.

12. A process for the preparation of a polypeptide according to any one of claims 8-11, said process comprising chemical synthesis, recombinant DNA technology or a combination of these methods.

13. A process for the preparation of a polynucleotide according to any one of claims 1-3, said process comprising chemical synthesis, recombinant DNA technology, polymerase claim reaction or a combination of these methods.

14. An antibody or a oligopeptide or a oligonucleotide or derivatives thereof which specifically recognizes and binds to a polypeptide as defined in claims 8-11.

15. A polynucleotide of any one of claims 1-4 or a polypeptide of any one of claims 8-11 for use in medicine.

16. Use of a polynucleotide of any one of claims 1-4 or a polypeptide of any one of claims 8-11 in the preparation of a pharmaceutical composition for the treatment of a disease resulting from shortage or lack or inactivity of said polypeptide.

17. A method of treatment of a disease resulting from shortage or lack, or inactivity of a polypeptide as defined in claims 8-11, said method comprising administration of a suitable amount of a polynucleotide of any one of claims 1-4 or a polypeptide.of any one of claims 8-11.

18. A method of treatment of a disease resulting from excessive activity or overexpression of a polypeptide as defined in claims 8-11, said method comprising administration of a suitable amount of an antibody or a oligopeptide or a oligonucleotide or derivatives thereof as defined in claim 14.

19. A method for identifying a substance capable of modulating the biological activity or expression of a polypeptide as defined in claims 8-11 in a cell, said method.comprising contacting the polypeptide or a functional derivative, a functional fragment or a functional, analog thereof, or a cell capable of expressing the polypeptide, with at least one compound or agent whose ability to modulate the biological activity or expression of said polypeptide, functional derivative, functional fragment or functional analog is sought to be investigated, and determining the change of the biological activity or the expression of said polypeptide, derivative or fragment caused by the substance.

20. The method of claim 19, further comprising formulating a pharmaceutical composition comprising as an active agent a substance which has been identified as a modulator or a derivative thereof.

21. An assay system for testing a substance for its capability of binding to or having functional effects on a polypeptide as defined in claims 8-11, said assay system comprising the polypeptide, or a functional derivative, a functional fragment or a functional analog thereof, or a cell capable of expressing the polypeptide or a functional derivative, a functional fragment or a functional analog and optionally means for determining a response caused by the substance.

22. A substance obtainable by a method as defined in claim 19 or 20, said substance being an agonist or antagonist of a polypeptide as defined in claims 8-11.

23. Use of a polynucleotide of any one of claims 1-4 or a fragment or derivative thereof for modulating the expression of a polypeptide as defined in claims 8-11 in a cell.

24. Use of a polynucleotide of any one of claims 14 in gene therapy.

25. Use of an antibody or a oligopeptide or a oligonucleotide or a derivative thereof as defined in claim 14 or of a polynucleotide or a fragment or derivative thereof of any one of claims 1-4 for diagnosis of a disease resulting from shortage or overexpression of a polypeptide a defined in claims 8-11.


SEQUENCE LISTING (5 sequences)

SEQ ID NO: 1 - DNA, 3943 bp, Homo sapiens
gacaggagacccttgcctgtagacagagctgcaggtttgaaggaaaagaccctgattctacttgatgtgagcaccaagaacccagtcaggacagtcaatgagaacttcctctctctgcagctggatccgtccatcattcatgatggctggctcgatttcctaagctccaagcgcttggtgaccctggcccggggactttcgcccgcctttctgcgcttcgggggcaaaaggaccgacttcctgcagttccagaacctgaggaacccggcgaaaagccgcgggggcccgggcccggattactatctcaaaaactatgaggatgacattgttcgaagtgatgttgccttagataaacagaaaggctgcaagattgcccagcaccctgatgttatgctggagctccaaagggagaaggcagctcagatgcatctggttcttctaaaggagcaattctccaatacttacagtaatctcatattaacagagccaaataactatcggaccatgcatggccgggcagtaaatggcagccagttgggaaaggattacatccagctgaagagcctgttgcagcccatccggatttattccagagccagcttatatggccctaatattgggcggccgaggaagaatgtcatcgccctcctagatggattcatgaaggtggcaggaagtacagtagatgcagttacctggcaacattgctacattgatggccgggtggtcaaggtgatggacttcctgaaaactcgcctgttagacacactctctgaccagattaggaaaattcagaaagtggttaatacatacactccaggaaagaagatttggcttgaaggtgtggtgaccacctcagctggaggcacaaacaatctatccgattcctatgctgcaggattcttatggttgaacactttaggaatgctggccaatcagggcattgatgtcgtgatacggcactcattttttgaccatggatacaatcacctcgtggaccagaattttaacccattaccagactactggctctctctcctctacaagcgcctgatcggccccaaagtcttggctgtgcatgtggctgggctccagcggaaaccacggcctggccgagtgatccgggacaaactaaggatttatgctcactgcacaaaccaccacaaccacaactacgttcgagggtccattacactttttatcatcaacttgcatcgakcaagaaagaaaatcaagctggctgggactctcagagacaagctggttcaccagtacctgctgcagccctatgggcaggagggcctaaagtccaagtcagtgcaactgaatggccagcccttagtgatggtggacgacgggaccctcccagaattgaagccccgcccccttcgggccggccggacattggtcatccctccagtcaccatgggcttttatgtggtcaagaatgtcaatgctttggcctgccgctaccgataagctatcctcacactcacggctaccagtgggcctgctgggctgcttccactcctccactccagtagtatcctctgttttcagacatcctagcaaccagcccctgctgccccatcctgctggaatcaacacagacttgctctccaaagagactaaatgtcatagcgtgatcttagcctaggtaggccacatccatcccaaaggaaaatgtagacatcacctgtacctatataaggataaaggcatgtgtatagagcagaatgtttcccttcatgtgcactatgaaaacgagctgacagcacactcccaggagaaatgtttccagacaactccccatgatcctgtcacacagcattataaccacaaatccaaaccttagcctgctgctgctgctgccctcagaggaagatgaggaaggaaaaaaactgggtggacctacaaaaacccatcctctcccaactccttcttctctgcctctttcttgctgctgccctgagttttttgacacatctctttccataggggagtaatgggtgtgtcagccctggcctgctgggagagctgtttgtatgatttcccggctgatgtatgagcgtgcgcatctgggttcctgacagtggcatccatcactggcagttcttctgggaagcgggtgcttcaaaagtaaaattacaatcacactccagatttggtaagaaggttctattcctctgtgaatccagattcccccagagttgtaatgggagtcaagtaacaatattcattgagtggagagcagtttattaggcacaacaaaaagtaatcatcattcttcatgttgctatgagggagagtttgagtacaaagagaaagcatactgaaacatcaggtacacacacacaccccaactggacaaagcaaattagacctctccaaaattaagagaatattaggggctctatagggtaagcctttaattgtttggttaactcaaatcattatttttaaaaaagaagaaaaaagtgtgaatcaaggtcatcactggaagacacaactgaatctaacctttttgcctcttcccaagtagcctatttgagctagaacaaaactttgttagccattttgggagagaatagggaatctagagaatgaagatctgcccaaaactatggaatggtaggtaggaagcttctgagttgggcaggtgtgaagtgggggatgaggacgttctatatgattcaaggggcatgagggtctttgccaatgagctacagctgaaatgactttcttttctggggatgtgattttctttctcaggataaatgacaggaatgatgcttttgttagaaggaggagagatttgacactgttccaagtgagacagtgatacaatttctgctgtttgtgaaaggacaggaatggggygggggcaaggcagggttgcctagggcagagactagggaggctgcctaagacgcacacggagttaaggatttgggccaagtctgcaaagtgagagatggaagggagattagaccaaagaggagggagagaattctgagcttggagaacggtggatttgggagagggaagctgactacctaattccaggaagcgaggggaccgggttttgacatgcttatcattaagcacaggaggaacagcatacagcagatgtactacagcgagcaagaaagggagagcccgaggaccaggctgcaccaggtcagtggctgtgctcagcatggaagcaactggagagagaggggcagaccctgagacygccctgcaaggctgcccagaagggacccgtttctctgggaccaggcacctcccactgaggcttcagctctgagagggcaggaaagtgaagtaccaagatgggggcggggcggggggtaggaaataagagaaagaagaaacagattgacaggccaaagtgaggaaaagagaggaaaagagaaatgagactaaaaggtcgttcccccaactgttaaaaatgtgtgcagatatcaacgtctcttctacatactggtacaggtgcgactgcagggccccctgatataacaagagtaaccaaaggtccctaagagcctggccctggggacctatggtttgctttgcgtccttagtaaccccatgataaaggggtactactgttatccccatttttcctacgaggcatggagaggatccatggctcgccccaggggcacccggggaaatgggttgccgagcgcgaaataatccagagcctgcccactcagccacaaggctcagcggctccacaggtccagacacctccttcacatctttgtaggttctgctcattcagaacagccagaactccactcaaacacactttctgtaaataagtgttgatttttttttactaaaccttgcagaatatgggtaattcctgcttcttttatctttctctgtgtattaaatgctgctctcacgagatttaagttttgtttatttttta

SEQ ID NO: 2 - protein, 492 aa, Homo sapiens
  MOD_RES (407): Ala or Ser
DRRPLPVDRAAGLKEKTLILLDVSTKNPVRTVNENFLSLQLDPSIIHDGWLDFLSSKRLVTLARGLSPAFLRFGGKRTDFLQFQNLRNPAKSRGGPGPDYYLKNYEDDIVRSDVALDKQKGCKIAQHPDVMLELQREKAAQMHLVLLKEQFSNTYSNLILTEPNNYRTMHGRAVNGSQLGKDYIQLKSLLQPIRIYSRASLYGPNIGRPRKNVIALLDGFMKVAGSTVDAVTWQHCYIDGRVVKVMDFLKTRLLDTLSDQIRKIQKVVNTYTPGKKIWLEGVVTTSAGGTNNLSDSYAAGFLWLNTLGMLANQGIDVVIRHSFFDHGYNHLVDQNFNPLPDYWLSLLYKRLIGPKVLAVHVAGLQRKPRPGRVIRDKLRIYAHCTNHHNHNYVRGSITLFIINLHRXRKKIKLAGTLRDKLVHQYLLQPYGQEGLKSKSVQLNGQPLVMVDDGTLPELKPRPLRAGRTLVIPPVTMGFYVVKNVNALACRYR

SEQ ID NO: 3 - DNA, 24 bp, Artificial Sequence
  Description of Artificial Sequence Primer
gacaggagacccttgcctgtagac

SEQ ID NO: 4 - DNA, 33 bp, Artificial Sequence
  Description of Artificial Sequence Primer
atagtcgagttatcggtagcggcaggccaaagc

SEQ ID NO: 5 - protein, 543 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI